(19) United States
(12) Patent Application Publication
(10) Pub. No.: US 2004/0265424 A1
(43) Pub. Date: Dec. 30, 2004

(54) Feed ration for finishing swine

(76) Inventors: Thomas M. Hayes, Wichita, KS (US); Rich Gallant, Wichita, KS (US); Kyle Swigart, Wichita, KS (US); Scott J. Eilert, Wichita, KS (US)

Correspondence Address: DORSEY & WHITNEY LLP, INTELLECTUAL PROPERTY DEPARTMENT, 50 SOUTH SIXTH STREET, MINNEAPOLIS, MN, 55402-1498, US

(21) Appl. No.: 10/607,837

(22) Filed: Jun. 27, 2003

(51) Int. Cl. A01K 1/00

(52) U.S. Cl. 426/2

(57) ABSTRACT

The present invention is a feed ration and a method of improving quality of a pork belly by including hydrogenated poultry fat as part of an animal's diet. The hydrogenated poultry fat may have an iodine value of between about 30 and about 60, and may be included in the feed ration on a daily basis during the final phases of the finishing diet.

### FIELD OF THE INVENTION

The present invention relates to methods of improving fat quality in an animal. More specifically, it relates to methods of improving fat quality by providing hydrogenated poultry fat for consumption by a finishing swine.

### BACKGROUND OF THE INVENTION

Pork belly quality is a concern for meat processors. The characteristics of a pork belly can influence the ability to process the pork belly into bacon. This processing typically involves several steps. After slaughter, the pork belly is harvested from the pig carcass and transferred from the slaughterhouse to a meat processing plant. At the meat processing plant, the belly is typically injected with flavoring solutions. Next, the belly is cooked to a certain temperature in a smokehouse. Then, the belly is placed into a molder, which presses each belly into a predetermined size and shape with a thickness and width that is constant for each belly. Finally, the molded belly is sliced into bacon.

One factor influencing the molding step is the quality of the pork belly. More specifically, both the firmness of the belly and its ability to retain shape impact the molding step. A pork belly that is not firm and is unable to retain shape will lose the predetermined shape after molding, making the slicing step difficult and resulting in low-quality, poorly-sliced bacon. A pork belly's firmness and ability to retain shape is increased by providing saturated fats in the diet of the pig. Fats consist of chains of carbon atoms. In saturated fats, those carbon chains are completely and uniformly filled with hydrogen atoms attached to the carbon atoms. Conversely, unsaturated fats lack some hydrogen atoms in their carbon chains. Saturated fat is therefore firmer than unsaturated fat, thus providing for a firmer belly that retains its post-molding shape.

Accordingly, there is a need in the art for a method of providing pork bellies with improved characteristics. More specifically, there is a need for a method of generating pork bellies having a higher concentration of saturated fats.

### SUMMARY OF THE INVENTION

The present invention, according to one embodiment, is a method of processing pork. The method includes feeding a daily feed ration to a pig, the daily ration including hydrogenated poultry fat, slaughtering the pig and harvesting a pork belly from the pig. The pork belly is then injected with flavoring and cooked to a predetermined temperature. The method further includes pressing the pork belly into a predetermined shape and slicing the pork belly into bacon slices. The present invention, according to another embodiment, is a method of feeding an animal. The method includes feeding the animal a daily feed ration including an amount of hydrogenated poultry fat.

The present invention, according to yet another embodiment, is a feed ration for raising finishing swine. The feed ration includes a grain and an amount of hydrogenated poultry fat, wherein the hydrogenated poultry fat has an iodine value of between about 30 and about 60.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a flow chart showing a method of improving pork belly quality, according to one embodiment of the present invention.

FIG. 2 is a flow chart showing a method of providing saturated fats in an animal's diet.

### DETAILED DESCRIPTION

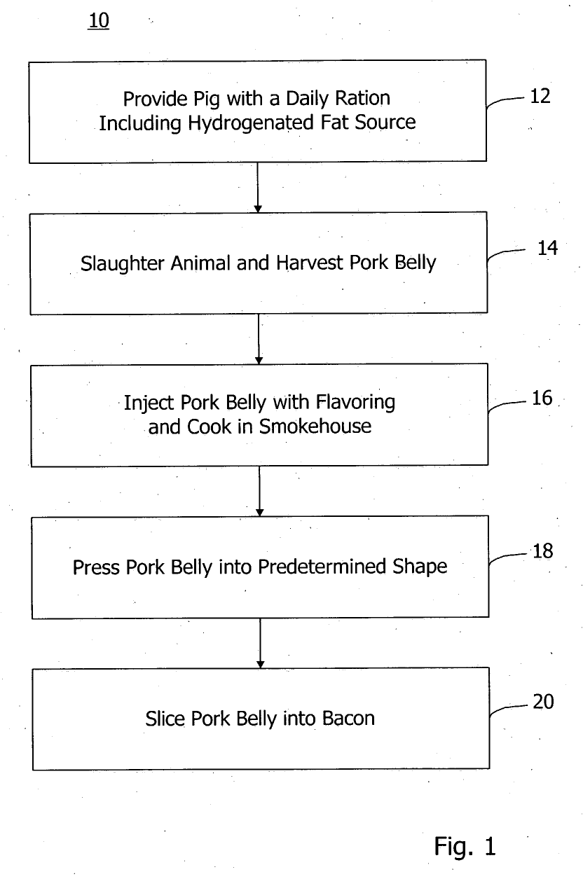

FIG. 1 is a flow chart showing one embodiment of a method 10 for producing an improved pork belly. The method 10 involves providing hydrogenated fats in a pig's daily rations (block 12). Next, the animal is slaughtered, and the pork belly is harvested from the carcass block (block 14). The harvested pork belly is then injected with flavoring, and cooked to a predetermined temperature in a smokehouse (block 16). Next, the pork belly is pressed into a predetermined shape by a molding apparatus (block 18). Finally, the molded pork belly is sliced into bacon (block 20).

In one embodiment, the hydrogenated fat provided in the pig's daily rations is hydrogenated poultry fat. In one embodiment, the poultry fat is subjected to sufficient hydrogenation to reduce its iodine value to between about 60 and about 30. In another embodiment, the iodine value is reduced to between about 45 and about 35. In one embodiment, the iodine value is reduced to about 40. In one embodiment, the hydrogenated poultry fat is a chicken fat or a turkey fat. The poultry fat may be hydrogenated using any hydrogenation process known in the art. The hydrogenation process increases the amount of saturated fats and reduces the amount of unsaturated fats. For example, in one embodiment, the poultry fat is hydrogenated using a known continuous hydrogenation process involving a Raney Ni catalyst. In one embodiment, the hydrogenated fat provided in the daily ration is a combination of hydrogenated poultry fat with another source of saturated fat.

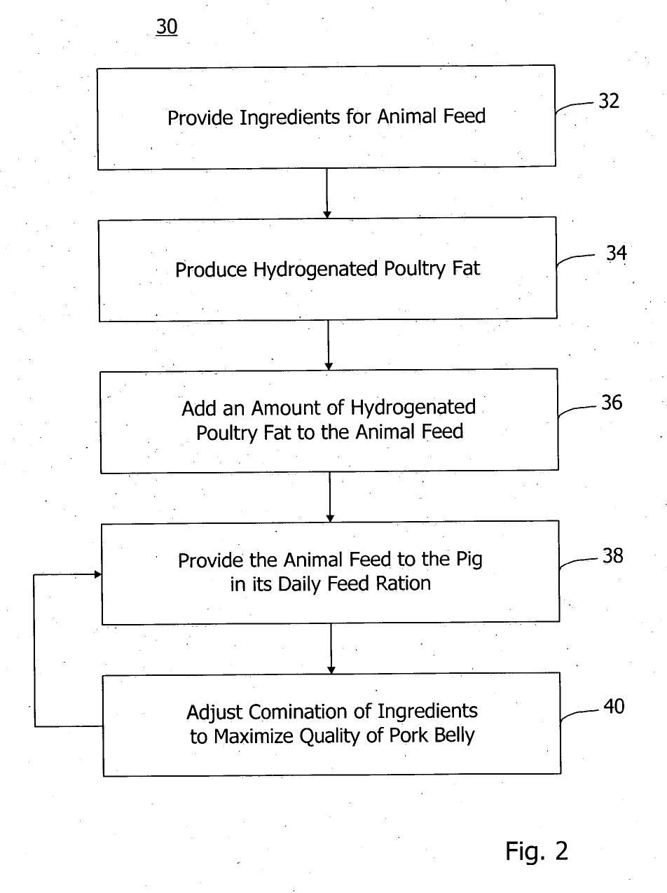

FIG. 2 is a flow chart showing a process 30 of producing a daily feed ration that includes hydrogenated poultry fat, according to one embodiment of the invention. As shown in FIG. 2, ingredients are provided to be included in the pig feed (block 32). For example, the pig feed may include ground corn, soybean meal, ground oats, and various other additives These other additive may include, for example, vitamins, minerals, antibiotics, and growth promoters (e.g., paylean). Poultry fat is hydrogenated to increase the concentration of saturated fat (block 34), and the hydrogenated fat is added to the animal feed (block 36). The animal feed is then provided to the pig in its daily ration (block 38). The ingredients in the feed, including the amount and types of saturated fats, can then be adjusted to maximize quality of the pork belly (block 40).

In one embodiment, the hydrogenated poultry fat is provided in the pig's daily rations throughout the entire process of raising the animal. In another embodiment, the hydrogenated poultry fat is provided in the pig's daily rations only during the final finishing phase. The finishing phase may include the three to four weeks prior to slaughter of the animal. In one embodiment, the animal is fed hydrogenated poultry fat after the pig has reached 100 pounds. In a further aspect, the animal is fed hydrogenated poultry fat after it has reached 150 pounds. Alternatively, the animal is fed hydrogenated poultry fat after it has reached 170 pounds. In one embodiment, a pig is provided with hydrogenated poultry fat for about eight weeks prior to slaughter. Alternatively, the pig is provided with saturated fats for about six weeks prior to slaughter. In a further alternative, the pig is provided with saturated fats for about three weeks prior to slaughter.

According to various embodiments of the present invention, the amount of hydrogenated poultry fat added to the animal feed varies. In one embodiment, the animal feed includes hydrogenated poultry fat in an amount of from about 0.5% to about 5% by weight. In one embodiment, the animal feed includes hydrogenated poultry fat in an amount of from about 1% to about 3% by weight. In one embodiment, the animal feed includes hydrogenated poultry fat in an amount of about 1.5%. In other embodiment, the weight percent of the hydrogenated poultry fat varies during the aging and growth of the animal. For example, in one embodiment, the amount of hydrogenated poultry fat added to the animal feed decreases as the animal nears a final market weight. In one embodiment, for example, the animal feed includes 3%, 2%, and then 1% hydrogenated fat by weight over the final three phases of the finishing diet.

The method of increasing the saturated fat content of animal meat is not limited to pork and can be applied to any animal from which an improved quality fat is desired.

### EXAMPLE

Three embodiments of the method of controlling the level of saturated fats in meat were compared to two diets low in saturated fats to examine the differences between each treatment based on (1) fatty acid profiles, (2) iodine values, (3) thickness, (4) flop distance, and (5) firmness of the resulting pork bellies. The fatty acid profiles include measurements of saturates, monounsaturates, polyunsaturates, and trans-fatty acids. Each measurement is a percentage of total weight.

Iodine value (“IV”) measures fat saturation, with a higher number indicating lower levels of saturated fat. When fat is treated with iodine, two atoms of iodine are absorbed at each double bond. Thus, a higher IV indicates more double bonds in the fat and consequently more unsaturated fat. For example, a saturated fat may have an IV of about 40 while an unsaturated fat may have an IV of about 75.

Thickness is a measure of pork belly thickness based on an average of three measurements taken at three predetermined locations on the pork belly including, a center point, and one point on each side of the center point halfway between the center point and each end. Flop distance is a measurement of the distance between the ends of a pork belly when the middle (a point halfway between the two ends) of the pork belly is draped over a designated rod. The greater the distance between the two ends, the firmer the belly.

Firmness is a measurement based on an equation developed by South Dakota State University and as provided in a paper entitled “The Effect of Dried Distillers Grain with Solubles in Swine Diets on Pork Muscle and Fat Quality,” presented at the 2002 Reciprocal Meat Conference at Michigan State University. The equation is:

$${cos}^{- 1} ( \frac{0.5 {L}^{2} - {D}^{2}}{0.5 {L}^{2}} )$$

where L is a measurement of pork belly length and D is the flop distance measurement.

The subject pigs were divided into five groups and each group was fed one of five different diets. The pigs were then slaughtered and the pork bellies tested for the above characteristics.

Testing

The five groups were fed one of five different diets. The control diet was a control diet containing corn and soybeans with no added fat. The unmodified poultry fat diet was another control diet containing poultry fat with iodine values ranging from 68 to 74. The tallow diet contained tallow with iodine values ranging from 40 to 50. The lard diet contained lard with iodine values ranging from 60 to 65. Finally, the hydrogenated poultry fat diet contained poultry fat that had been hydrogenated with a target iodine value of 40.

Table 1 provides iodine values and fatty acid profiles obtained from samples of the fat sources used in the above diets.

TABLE 1
Fatty Acid Profiles of the Fat Sources Used

| | Unmodified Poultry | | | Hydrogenated |
|---|---|---|---|---|
| | Fat | Tallow | Lard | Poultry Fat |
| Iodine Values | 69.02 | 50.18 | 63.63 | 42.04 |
| Saturates | 36.82 | 47.63 | 38.30 | 57.62 |
| Monounsaturates | 43.39 | 44.17 | 46.56 | 34.88 |
| Polyunsaturates | 18.70 | 7.02 | 14.39 | 6.63 |
| Trans Fatty | 4.36 | 4.17 | 1.00 | 12.73 |
| Acids | | | | |

These samples of the fat sources indicated that hydrogenated poultry fat had the highest level of saturated fat of the five diets examined, followed closely by tallow.

Results

Table 2 provides belly quality data collected from the pork bellies of the test animals after slaughter. The three measurements were thickness, flop distance, and a firmness score. As previously discussed, thickness is the average thickness of each pork belly based on measurements taken at three predetermined locations on each belly. Flop distance is the distance between two ends of a pork belly when the belly is placed over a suspended bar. The firmness score is based on an equation that combines flop distance with belly length and is regressed with the iodine value of the pork belly.

TABLE 2
Belly Quality Data

| | Thickness | Flop Distance | Firmness Score |
|---|---|---|---|
| Control Diet | 1.13 | 10.37 | 45.6 |
| Unmodified Poultry | 1.08 | 7.98 | 35.4 |
| Fat Diet | | | |
| Tallow Diet | 1.15 | 8.73 | 38.4 |
| Lard Diet | 1.08 | 7.48 | 33.1 |
| Hydrogenated Poultry Fat | 1.05 | 9.13 | 40.0 |
| Diet | | | |

There was no significant variation in pork belly thickness across the treatments. As seen in the second column, pork bellies from pigs fed the tallow diet and the hydrogenated poultry fat diet exhibited flop distances greater than those of pigs fed unmodified poultry fat or lard diets. In addition, the firmness score was also similar for pork bellies originating from the tallow and hydrogenated poultry fat diets, both having significantly higher scores than the pork bellies from pigs fed lard or unmodified poultry fat.

Table 3 provides iodine values and fatty acid profiles collected from the pork bellies of the test animals after slaughter.

TABLE 3
Fatty Acid Profiles of the Pork Belly Fat Samples

| | | | | | Trans- |
|---|---|---|---|---|---|
| | Iodine | | Mono- | Poly- | Fatty |
| | Value | Saturates | Unsaturated | Unsaturated | Acids |
| Control | 62.30 ± 2.13 | 37.97 ± 1.74 | 49.11 ± 1.18 | 12.34 ± 1.10 | 0.53 ± 0.10 |
| Tallow | 65.22 ± 1.47 | 35.53 ± 1.38 | 50.00 ± 0.92 | 13.56 ± 0.72 | 1.07 ± 0.12 |
| Hydrogenated | 64.51 ± 1.59 | 36.79 ± 1.36 | 48.64 ± 1.09 | 13.77 ± 0.84 | 2.41 ± 0.60 |
| Poultry Fat | | | | | |
| Lard | 68.59 ± 2.48 | 33.69 ± 1.95 | 49.33 ± 1.17 | 15.82 ± 1.16 | 0.71 ± 0.17 |
| Unmodified | 68.91 ± 1.60 | 31.51 ± 1.60 | 47.65 ± 1.36 | 16.97 ± 1.16 | 1.28 ± 0.23 |
| Poultry Fat | | | | | |

As expected, pigs fed fats with lower iodine values produced pork belly fat with lower iodine values. The low iodine values of the pork belly fat originating from pigs fed hydrogenated poultry fat and tallow reflected a higher level of saturation and thus firmer fat. The higher saturates and lower polyunsaturated fats for the same pork bellies reflected the same characteristics. The trans-fatty acids data showed that pigs fed hydrogenated poultry fat produced belly fat higher in trans-fatty acids than the other treatments.

We claim:

1. A method of raising finishing swine to improve belly firmness, the method comprising:
feeding a daily feed ration to a pig, the daily ration including hydrogenated poultry fat;
slaughtering the pig and harvesting a pork belly from the pig;
injecting the pork belly with flavoring and cooking the pork belly to a predetermined temperature;
pressing the pork belly into a predetermined shape; and
slicing the pork belly into bacon slices.

2. The method of claim 1 wherein the hydrogenated poultry fat is hydrogenated chicken fat.

3. The method of claim 1 wherein the feeding step is performed on a daily basis for between about three and about eight weeks prior to slaughtering the pig.

4. The method of claim 1 wherein the feeding step is performed on a daily basis after the pig has reached a weight of between about 100 and about 170 pounds.

5. The method of claim 1 wherein the hydrogenated poultry fat has an iodine value of between about 60 and about 30.

6. The method of claim 1 wherein the hydrogenated poultry fat has an iodine value of less than about 45.

7. The method of claim 1 wherein the feed ration further includes a second saturated fat source.

8. The method of claim 7 wherein the second saturated fat source is tallow.

9. A method of feeding an animal comprising feeding the animal a daily feed ration including an amount of hydrogenated poultry fat, wherein the hydrogenated poultry fat has an iodine value of between about 60 and about 30.

10. The method of claim 9 wherein the animal is a pig.

11. The method of claim 10 wherein the hydrogenated poultry fat is feed to the pig on a daily basis for at least three weeks before the pig is slaughtered.

12. The method of claim 10 wherein the hydrogenated poultry fat is feed to the pig on a daily basis after the pig weighs at least 170 pounds.

13. The method of claim 9 wherein the only fat source in the daily feed ration is hydrogenated poultry fat.

14. The method of claim 9 wherein the hydrogenated poultry fat has an iodine value of less than about 45.

15. The method of claim 9 wherein the feed ration further includes a second saturated fat source.

16. A feed ration for feeding finishing swine, the feed ration comprising a grain and an amount of hydrogenated poultry fat, wherein the hydrogenated poultry fat has an iodine value of between about 30 and, about 60.

17. The feed ration of claim 16 wherein the feed ration has an iodine value of about 40.

18. The feed ration of claim 17 wherein the hydrogenated poultry fat is hydrogenated chicken fat.

19. The feed ration of claim 16 further including a second saturated fat source.

20. The feed ration of claim 19 wherein the second saturated fat source is tallow.
